(19) United States
(12) Patent Application Publication
(10) Pub. No.: US 2010/0121411 A1
(43) Pub. Date: May 13, 2010

(54) Tinnitus Suppressing Cochlear Implant

(75) Inventors: Ingeborg Hochmair, Axams (AT); Martin Zimmerling, Patsch (AT); Clemens M. Zierhofer, Kundl (AT); Katrien Vermeire, Meiming (AT)

Correspondence Address: Sunstein Kann Murphy & Timbers LLP, 125 SUMMER STREET, BOSTON, MA, 02110-1618, US

(73) Assignee: MED-EL Elektromedizinische Geraete GmbH, Innsbruck (AT)

(21) Appl. No.: 12/615,731

(22) Filed: Nov. 10, 2009

Related U.S. Application Data
(63) Continuation-in-part of application No. 11/671,636, filed on Feb. 6, 2007.
(60) Provisional application No. 60/765,775, filed on Feb. 7, 2006.

(51) Int. Cl. A61N 1/36; A61F 11/00

(52) U.S. Cl. 607/55

(57) ABSTRACT

An implantable device and corresponding method for suppression of tinnitus are described. An implantable signal processing module develops a stimulation signal for application to audio sensing tissue of the user. The signal processing module includes a tinnitus suppression mode in which the stimulation signal is unrelated to environmental sound near the user.

### CROSS-REFERENCE TO RELATED APPLICATIONS

This application is a continuation-in-part of U.S. application Ser. No. 11/671,636, filed Feb. 6, 2007, which in turn claims priority from U.S. Provisional Application 60/765,775, filed Feb. 7, 2006. Each of the above-described applications is incorporated herein by reference, in their entirety.

### FIELD OF INVENTION

The invention relates to an implantable device for persons suffering from tinnitus.

### BACKGROUND ART

A normal ear transmits sounds as shown in FIG. 1 through the outer ear 101 to the eardrum 102, which moves the bones of the middle ear 103, which in turn excites the cochlea 104. The cochlea 104 includes an upper channel known as the scala vestibuli 105 and a lower channel known as the scala tympani 106, which are connected by the cochlear duct 107. In response to received sounds transmitted by the middle ear 103, the fluid filled scala vestibuli 105 and scala tympani 106 function as a transducer to transmit waves to generate electric pulses that are transmitted to the cochlear nerve 113, and ultimately to the brain.

Some persons have partial or full loss of normal sensorineural hearing. Cochlear implant systems have been developed to overcome this by directly stimulating the user's cochlea 104. A typical system may include an external microphone that provides an audio signal input to an external signal processing stage (not shown in FIG. 1) where various signal processing schemes can be implemented. The processed signal is then converted into a digital data format, such as a sequence of data frames, for transmission into stimulator 108. Besides extracting the audio information, the stimulator 108 also performs additional signal processing such as error correction, pulse formation, etc., and produces a stimulation signal (based on the extracted audio information) that is sent through connected wires 109 to an implanted electrode carrier 110. Typically, this electrode carrier 110 includes multiple electrodes on its surface that provide selective stimulation of the cochlea 104.

Existing cochlear implant systems need to deliver electrical power from outside the body through the skin to satisfy the power requirements of the implanted portion of the system. FIG. 1 shows a typical arrangement based on inductive coupling through the skin to transfer both the required electrical power and the processed audio information. As shown in FIG. 1, an external primary coil 111 (coupled to the external signal processor) is placed on the skin adjacent to a subcutaneous secondary coil 112 (connected to the stimulator 108). Often, a magnet in the external coil structure interacts a corresponding magnet in the subcutaneous secondary coil structure. This arrangement inductively couples a radio frequency (rf) electrical signal to the stimulator 108. The stimulator 108 is able to extract from the rf signal both the audio information for the implanted portion of the system and a power component to power the implanted system.

Many profoundly deaf persons do not need or want a cochlear implant system to improve their communication skills. The implant-aided input is very different from the input via a normal ear, and therefore many profoundly deaf users do not bother to undergo any training. Therefore they do not see any advantage and soon terminate the use of their cochlear implant. In addition, cochlear implants are normally not considered for subjects suffering from unilateral hearing loss (hearing loss on one side).

Besides hearing loss, another rather depressing hearing-related affliction is tinnitus. Tinnitus is defined by the perception of a continuous ringing or beating sound without external source. This sensation can be extremely annoying and often interferes with normal daily activities including sleep.

### SUMMARY OF THE INVENTION

Embodiments of the present invention include an implantable device for suppression of tinnitus. An implantable stimulator module develops a stimulation signal for application to audio sensing tissue of the user. The stimulator module includes a tinnitus suppression mode in which the stimulation signal is unrelated to environmental sound near the user.

In further embodiments, the stimulation signal may be significantly imperceptible to the user. The device may be a cochlear implant, for example, wherein the stimulation signal is an electrical stimulation signal and may be further adapted to stimulate the scala tympani and/or scala vestibuli of the user. The device may include an implantable stimulator which may be atraumatically insertable so as to preserve residual hearing in the implanted ear. Embodiments may also include an acoustic-mechanical stimulation module for developing an acoustic-mechanical stimulation signal such that the implanted ear receives both an electrical stimulation signal and an acoustic-mechanical stimulation signal.

FIG. 2 shows an implantable system according to one specific embodiment starting from the prior art system of FIG. 1.

The electrical stimulation signal may include sequences of electrical pulses at or near a threshold level of detectability to stimulate the audio sensing tissue. The electrical pulses may have amplitudes according to a CIS-strategy threshold and may occur at rates between 10 and 10,000 pulses per second.

In other embodiments, the device may be a brainstem implant. In other embodiments, the stimulation signal may be mechanical, for example, the device may be a middle ear implant such as a floating mass transducer.

In any such embodiment, the tinnitus suppression mode may be user controllable and/or software controllable, for example, controlled by time such that the tinnitus suppression mode is time dependent. The signal processing module may further provide signal processing to provide sound localization information.

Embodiments of the present invention also include a method for tinnitus suppression. In such embodiments, a stimulation signal unrelated to environmental sound near the user is applied to audio sensing tissue of a user. The stimulation signal may not be significantly perceptible to the user. In other embodiments, the stimulation signal may be strong enough to cause a perception in the patient, which may be faded and/or masked by the user's natural signal processing.

In some embodiments, the stimulation signal is an electrical stimulation signal provided by a cochlear implant. Further, the audio sensing tissue may include the scala tympani and/or the scala vestibuli of the user. The electrical stimulation signal may be applied using an atraumatically-inserted electrode which preserves residual hearing in the implanted ear.

Embodiments may also include providing acoustic mechanical stimulation to the implanted ear, such that the implanted ear receives both an electrical stimulation signal and an acoustic-mechanical stimulation signal. Applying the electrical stimulation signal may include applying sequences of electric pulses having amplitudes according to a CIS-strategy, for example, at rates between 10 and 10,000 pulses per second.

In some embodiments, the stimulating may be produced by a brainstem implant or by a middle ear implant such as a floating mass transducer or a bone conducting device.

The stimulating may be user controllable and/or software controllable, for example, to be time dependent. The stimulation signal may further provide sound localization information.

In accordance with an embodiment of the invention, an implantable device for suppression of tinnitus includes an implantable simulator module for developing a stimulation signal for application to audio sensing tissue of a user. The stimulator module includes at least a tinnitus suppression mode in which the stimulation signal includes a tinnitus suppression signal, the stimulator module including a memory device for storing at least one parameter of the tinnitus suppression signal. The stimulator module generates the tinnitus suppression signal as a function of the at least one parameter stored in the memory device, independent of data received substantially simultaneously by the stimulator module, if any.

In accordance with related embodiments of the invention, the stimulator module may generate the tinnitus suppression signal without contemporaneously interfacing with an external device. The stimulator module may generate the tinnitus suppression signal independent of acoustic signals of the nearby environment.

In accordance with other related embodiment of the invention, the implantable device may include a signal processing module operatively coupled to the stimulator module, the signal processing module processing an input acoustic audio signal representative of environmental sound to form a processed audio signal for providing to the stimulator module. The stimulator module may include at least one mode where the stimulation signal is developed based, at least in part, on the processed audio signal, wherein the stimulator module generates the tinnitus suppression signal independent of data received from the signal processing module. The signal processing module may be adapted to be worn external to the user, or may be adapted for implantation into the user.

In accordance with still further embodiments of the invention, the device may be a cochlear implant and the stimulation signal is an electrical stimulation signal. The tinnitus suppression signal may include biphasic pulses for sequentially stimulating at least one electrode, wherein the biphasic pulses are non-overlapping in time. The device may further include an electrode array, wherein the tinnitus suppression signal includes pulses for simultaneously stimulating two or more electrodes. The tinnitus suppression signal may include stimulation pulses having a time varying envelope based on a noise modulation function.

In accordance with yet further embodiments of the invention, the device may be a brainstem implant, a middle ear implant, or a bone conducting implant. The tinnitus suppression signal may not be significantly perceptible to the user, or may be masked by the natural signal processing of the user.

In accordance with another embodiment of the invention a method of suppressing tinnitus includes generating, by a stimulator module of an implanted prosthesis, a stimulation signal for application to audio sensing tissue of a user. The stimulator module includes at least a tinnitus suppression mode in which the stimulation signal includes a tinnitus suppression signal. The stimulator module generates the tinnitus suppression signal as a function of at least one parameter stored in stimulator memory, independent of any data received substantially simultaneously by the stimulator, if any.

In accordance with related embodiments of the invention, generating the tinnitus suppression signal may occur without contemporaneously interfacing with an external device. The tinnitus suppression signal may be unrelated to acoustic signals of the nearby environment.

In accordance with further related embodiments of the invention, generating the tinnitus signal may occur independent of data received by the stimulator from a signal processing module, the signal processing module processing an audio signal representative of environmental sound to form a processed audio signal for providing to the stimulator module. The stimulator module may include at least one mode where the stimulation signal is developed based, at least in part, on the processed audio signal. The signal processing module may be external to the user, or may be implanted into the user.

In accordance with still further embodiments of the invention, the tinnitus suppression signal may include biphasic pulses for sequentially stimulating at least one electrode, the biphasic pulses are non-overlapping in time. The tinnitus suppression signal may include pulses for simultaneously stimulating two or more electrodes. The tinnitus suppression signal may include stimulation pulses having a time varying envelope based on a noise modulation function. The tinnitus suppression signal when applied to the audio sensing tissue of the user may or may not be significantly perceptible. The tinnitus suppression signal may be masked by the natural signal processing of the user. The prosthesis may be a cochlear implant, a brainstem implant, a middle ear implant and/or a bone conducting implant.

In accordance with another embodiment of the invention, a computer program product in a computer readable storage medium is presented. The product includes program code for producing a data signal for an implanted audio prosthesis including program code for generating, in a stimulator module of the implanted prosthesis, a stimulation signal for application to audio sensing tissue of a user. The stimulator module includes at least a tinnitus suppression mode in which the stimulation signal includes a tinnitus suppression signal. The tinnitus suppression signal is generated as a function of at least one parameter stored in stimulator memory, independent of any data received substantially simultaneously by the stimulator module, if any.

In accordance with related embodiments, the program code for generating the stimulation signal may generate the tinnitus suppression signal without contemporaneously interfacing with an external device. The program code for generating the stimulation signal may generate the tinnitus signal independent of data received by the stimulator from a signal processing module, the signal processing module processing an audio signal representative of environmental sound to form a processed audio signal for providing to the stimulator module. The stimulator module may include at least one mode where the stimulation signal is developed based, at least in part, on the processed audio signal.

### BRIEF DESCRIPTION OF THE DRAWINGS

FIG. 1 shows the ear structure of a human ear and a typical cochlear implant system according to the prior art.

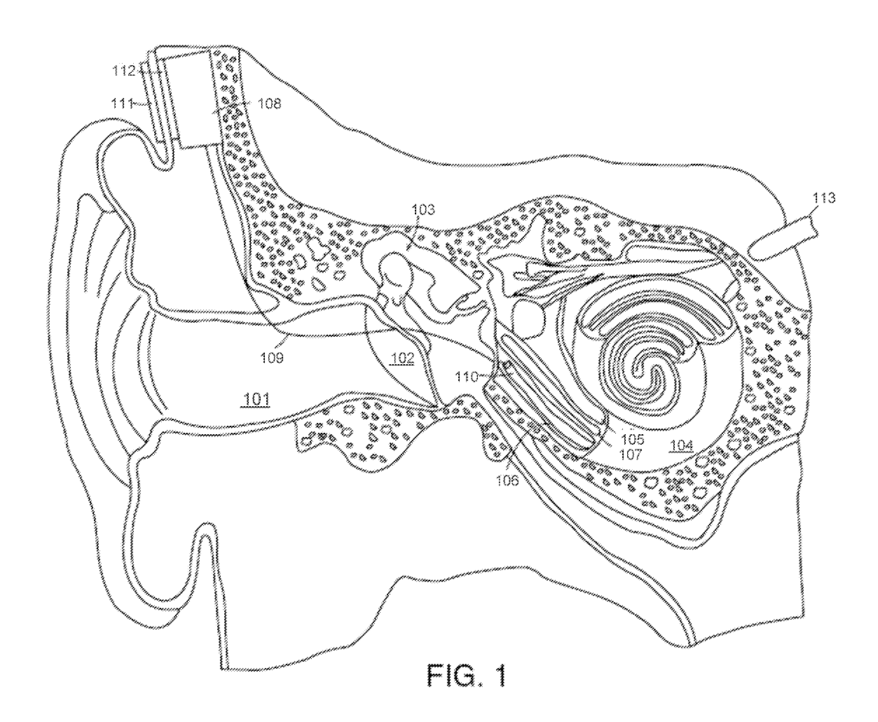

FIG. 2 shows an implantable system according to one specific embodiment of the present invention, starting from the prior art system of FIG. 1.

FIG. 3 shows a brainstem implant according to an alternative embodiment of the present invention.

### DETAILED DESCRIPTION OF SPECIFIC EMBODIMENTS

As used herein, the term “hearing implant” includes any type of hearing system having implantable parts, such as, without limitation, cochlear implants (CI), brainstem implants, bone conducting implants, and middle ear implants (MEI).

When stimulating, hearing implants can suppress some forms of tinnitus such as peripheral origin-tinnitus, i.e. tinnitus connected to hearing problems. Both mechanical (acoustic) and electrical stimulation have been found to provide such benefit. Specifically, unilateral tinnitus resulting from unilateral cochlear profound sensory-neural hearing loss (SNHL) can be treated with cochlear implantation, which also typically improves the overall quality of hearing in a significant way. As explained above, unilateral deafness has not previously been an indication for a cochlear implant. Thus, embodiments of the present invention include a cochlear implant that restores hearing by changing cortical activity by electrical stimulation of the auditory cortex or auditory nerve. Tinnitus suppression may be realized by use of comparatively low level stimulation (“background stimulation”) at or near the hearing threshold. The stimulation may or may not be at a perceptible level. If the stimulation is perceptible, it may be faded and/or masked by the user's natural signal processing.

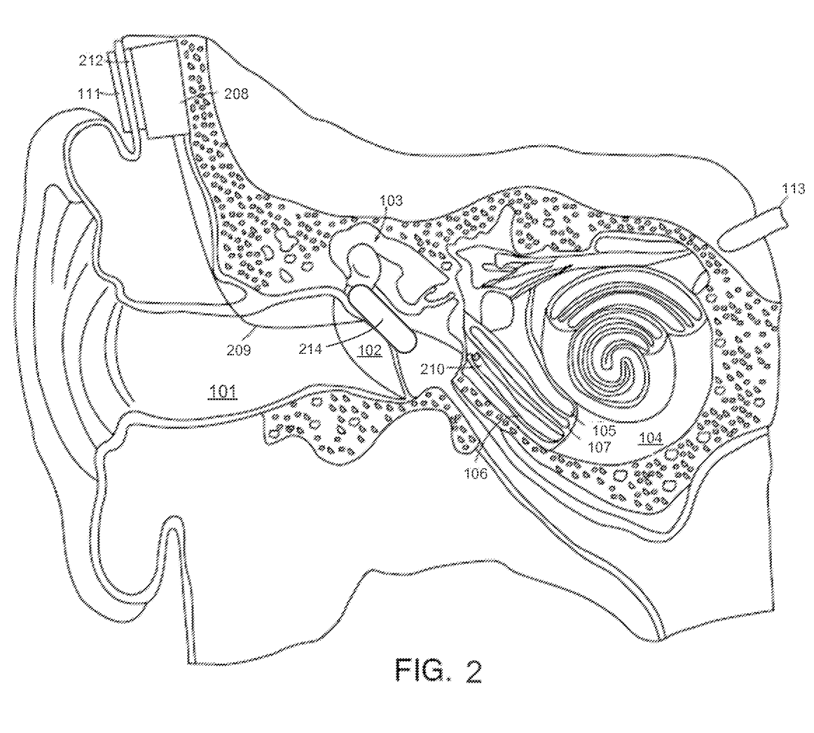

In specific embodiments, a cochlear implant may provide a stimulator signal that stimulates the scala tympani and/or scala vestibuli of the user. One specific embodiment can be explained with reference to the cochlear implant system shown in FIG. 2. A stimulation signal is developed by an implantable stimulator module 208. An electrode array 210 is coupled to the stimulator module 208 for applying the stimulation signal to audio sensing tissue of a user such as the scala vestibuli 105 and scala tympani 106 of the cochlea 104. The stimulator module 208 may have various operating modes which may or may not include a normal operation mode in which the stimulation signal applied to the audio sensing tissue is representative of the environmental sounds around the user.

Whether or not a normal mode is available, embodiments of the present invention include a tinnitus suppression mode in which the stimulation signal includes a tinnitus suppression signal that is unrelated to environmental sound near the user. In some specific embodiments, it may not be necessary to generate specific hearing sensations for tinnitus suppression, rather some low-level background stimulation appears to be sufficient. Thus, the tinnitus suppression mode in specific embodiments may be the result of switching off the microphone input, and/or providing some background stimulation at or near some threshold detection level of stimulation, such as provided by a CIS-type strategy as implemented, for example, in the Pulsar and Sonata implants by MED-EL of Innsbruck, Austria. The electrical stimulation signal may be in the form of CIS-type pulses, for example, at a rate of 10 to 10,000 pulses per second, at a pulse amplitude near the CIS threshold.

The electrode array 210 may be inserted relatively deeply in order to reach the low frequency response region of the cochlea 104 and thereby cover the entire frequency range. This may be especially important if the tinnitus effects occur in the low frequencies. In addition, use of such a deep insertion electrode array 210 in a cochlear implant for a unilaterally deaf person may also provide sound localization information so to restore or partially restore directional hearing for those persons in which the working ear is confined to low frequencies. In addition or alternatively, the electrode array 210 may also be suitable for atraumatic insertion so as to preserve some or all of the residual hearing in the implanted ear, which is often not a consideration in a cochlear implant for a totally deaf patient.

Some embodiments not only provide tinnitus suppression, but may also fully or partially restore bilateral hearing. This advantage is related to the use of an electrode array 210 adapted for deep insertion, in order to efficiently exploit the inputs from the normal ear and from the stimulator 208. The improvement in bilateral hearing may in turn improve speech understanding in noisy environments. In addition, directional hearing may be fully or partially restored. These effects generally may improve over time as the user accumulates experience with the device, especially if hearing on the non-implanted ear is also compromised.

Since tinnitus can be especially annoying during sleep time, a totally implantable cochlear implant can be of great advantage (albeit not absolutely necessary) to stimulate without having to wear an external part, which would have to be securely fastened and which might be uncomfortable. The tinnitus suppression mode may be user controllable and/or software controllable. For example, an embodiment could include a clock function to switch off sound input for a selected time (while retaining background stimulation for tinnitus suppression) such as at night for sleeping, or to optionally switch off stimulation after some time, and/or to set an alarm.

In illustrative embodiments, an implantable device for suppression of tinnitus is presented that is operable without contemporaneously interfacing with an external device. The implant may serve, without limitation, to function as a fully implantable cochlear implant, while in other embodiments the implant may serve only to suppress tinnitus suppression. For example, in many cases patients receiving a cochlear implant on one ear have normal or almost normal hearing on their contralateral ear. Thus, electrostimulation may serve exclusively for tinnitus suppression, and not for restoration of hearing. In such cases, tinnitus electrostimulation may be achieved using an artificial stimulation pattern that does not rely on acoustic signals from the environment, and which does not require complex stages for audio signal processing. As a consequence, a system for tinnitus suppression may be implemented much simpler and with significantly reduced power consumption as compared to a standard cochlear implant. For example, the electrode array for tinnitus suppression may require less active electrodes in the cochlea as compared to a standard cochlear implant.

As in above-described embodiments, the implant may include a stimulator module for stimulating an electrode array. The stimulator module may include, without limitation, a microprocessor, various circuitry and/or software. The implant may include an inductive link for transcutaneously interfacing with an external device when desired. The inductive link may be used to send and/or receive data, power or control signals. The implant may further include a microphone and/or a rechargeable battery that may be recharged via the inductive link.

In illustrative embodiments, the stimulator module includes a memory device for storing at least one parameter associated with the tinnitus suppression pattern. The memory device may be, without limitation, Read Only Memory (ROM), Erasable Programmable Read-Only Memory (EPROM), Random Access Memory (RAM), EEPROM and/or Flash-Programmable RAM. The parameter(s) stored in memory is used by the stimulator module to generate stimulation signals for tinnitus treatment which do not depend on the acoustic signal from the surrounding environment. Furthermore, the stimulation signals generated by the stimulator may be generated independent of any data received substantially simultaneously by the stimulator module, such as from, without limitation: an external device; or a signal processing module of a totally implantable cochlear implant that, at least in part, processes an audio signal, for example, received from an internal microphone.

The stimulation parameters used to generate the tinnitus suppression signal may be, for example, downloaded into memory at the factory, and/or previously downloaded from an external device via the inductive link (when the patient is wearing the external device). In various embodiments, the implant is not operatively coupled to an external device when generating the tinnitus suppression signal. Alternatively, the patient may be relying on, and wearing, the external device for purposes other than generating the tinnitus suppression signal (such as power transmission) when generating the tinnitus suppression signal.

In various embodiments, the tinnitus suppression signal may be automatically generated by periodic sequences of biphasic stimulation on each electrode. For each electrode, the pulse amplitudes may be adjusted for maximum tinnitus suppression.

In further embodiments, the envelopes of the automatically generated stimulation pulses in each channel are not constant, but time varying according to particular amplitude modulation functions. Such functions include, without limitation, sinusoids and rectangles.

In still other embodiments, the envelopes of the automatically generated stimulation pulses in each channel are not constant, but time varying according to noise-like modulation functions. The amplitude density functions of the noise functions may be adjusted for maximum tinnitus suppression.

The tinnitus suppression signals described above may be applied non-overlapping in time, similar to the pulses used for CIS. In still another embodiment, the tinnitus suppression signals may be applied using, at least in part, simultaneous stimulation pulses, as described, for example, in U.S. Pat. No. 6,594,525, which is incorporated herein by reference it its entirety. Simultaneous stimulation generally allows for an increase in the phase durations of the pulses and also a reduction in pulse amplitudes. Thus a better stimulation efficacy can be achieved.

If the subject retains some residual hearing in the implanted ear, the device also can be used together with an acoustic-mechanical stimulation module 214 to result in improved hearing quality and improved sound localization capability based on the application of both an electrical stimulation signal from the electrode carrier 210 and an acoustic mechanical stimulation signal from the acoustic mechanical stimulation module 214. The acoustic mechanical stimulation module 214 mechanically drives the ossicular chain, which in turn stimulates the cochlea 104. An acoustic mechanical stimulation module 214 in the specific form of a middle ear implant based on a floating mass transducer is further described, for example, in U.S. Pat. Nos. 5,913,815; 5,897,486; 5,624,376; 5,554,096; 5,456,654; 5,800,336; 5,857,958; and 6,475,134, each of which is incorporated herein by reference.

An alternative embodiment may have an acoustic mechanical stimulation module 214 with a tinnitus suppression mode, without any implanted electrode stimulation system so that only acoustic-mechanical stimulation is provided. Thus, specific embodiments may be in the form of a bone conduction system or a Middle Ear Implant (MEI) such as a “Soundbridge” (and its derivations) in which the stimulation is acoustic-mechanical via a “floating mass transducer.” The advantages gained by the patient are similar to a cochlear implant embodiment, and built-in features could be very similar as well. An MEI is often designed for moderately hearing impaired patients, who usually try to use conventional hearing aids thus avoiding surgery necessary for the MEI. However, because of the improved sound quality and the ability to suppress tinnitus during sleep, such a device may be more readily accepted.

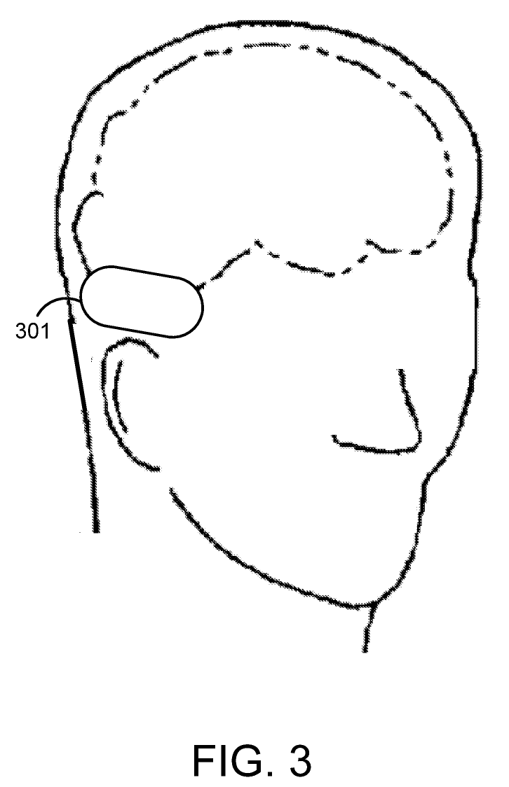

FIG. 3 shows another embodiment for tinnitus suppression in the specific form of a brainstem implant 301. In other embodiments, instead of a deep insertion scala tympani electrode, other stimulating means may be used, such as split electrodes (to stimulate the scala vestibuli), brainstem electrodes, floating mass transducer (at the ossicles or at the round window), and/or a bone bridge.

The present invention may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof.

Computer program logic implementing all or part of the functionality previously described herein may be embodied in various forms, including, but in no way limited to, a source code form, a computer executable form, and various intermediate forms (e.g., forms generated by an assembler, compiler, linker, or locator.) Source code may include a series of computer program instructions implemented in any of various programming languages (e.g., an object code, an assembly language, or a high-level language such as Fortran, C, C++, JAVA, or HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies, networking technologies, and internetworking technologies. The computer program may be distributed in any form as a removable storage medium with accompanying printed or electronic documentation (e.g., shrink wrapped software or a magnetic tape), preloaded with a computer system (e.g., on system ROM or fixed disk), or distributed from a server or electronic bulletin board over the communication system (e.g., the Internet or World Wide Web.)

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL.)

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable device for suppression of tinnitus, the device comprising:
an implantable simulator module for developing a stimulation signal for application to audio sensing tissue of a user, the stimulator module including at least a tinnitus suppression mode in which the stimulation signal includes a tinnitus suppression signal, the stimulator module including a memory device for storing at least one parameter of the tinnitus suppression signal,
wherein the stimulator module generates the tinnitus suppression signal as a function of the at least one parameter stored in the memory device, independent of data received substantially simultaneously by the stimulator module, if any.

2. The device according to claim 1, wherein the stimulator module generates the tinnitus suppression signal without contemporaneously interfacing with an external device.

3. The device according to claim 1, further comprising a signal processing module operatively coupled to the stimulator module, the signal processing module processing an input acoustic audio signal representative of environmental sound to form a processed audio signal for providing to the stimulator module; the stimulator module including at least one mode where the stimulation signal is developed based, at least in part, on the processed audio signal, wherein the stimulator module generates the tinnitus suppression signal independent of data received from the signal processing module.

4. The device according to claim 3, wherein the signal processing module is adapted to be worn external to the user.

5. The device according to claim 3, wherein the signal processing module is adapted for implantation into the user.

6. The device according to claim 1, where the stimulator module generates the tinnitus suppression signal independent of acoustic signals of the nearby environment.

7. The device according to claim 1, wherein the device is a cochlear implant and the stimulation signal is an electrical stimulation signal.

8. The device according to claim 7, wherein the tinnitus suppression signal includes biphasic pulses for sequentially stimulating at least one electrode, wherein the biphasic pulses are non-overlapping in time.

9. The device according to claim 7, further comprising an electrode array, wherein the tinnitus suppression signal includes pulses for simultaneously stimulating two or more electrodes.

10. The device according to claim 7, wherein the tinnitus suppression signal includes stimulation pulses having a time varying envelope based on a noise modulation function.

11. The device according to claim 1, where the device is a brainstem implant.

12. The device according to claim 1, wherein the device is a middle ear implant.

13. The device according to claim 1, wherein the device is a bone conducting implant.

14. The device according to claim 1, wherein the tinnitus suppression signal is not significantly perceptible to the user.

15. The device according to claim 1, wherein the tinnitus suppression signal is masked by the natural signal processing of the user.

16. A method of suppressing tinnitus, the method comprising:
generating, by a stimulator module of an implanted prosthesis, a stimulation signal for application to audio sensing tissue of a user, the stimulator module including at least a tinnitus suppression mode in which the stimulation signal includes a tinnitus suppression signal, the stimulator module generating the tinnitus suppression signal as a function of at least one parameter stored in stimulator memory, independent of any data received substantially simultaneously by the stimulator module, if any.

17. The method according to claim 16, wherein generating the tinnitus suppression signal occurs without contemporaneously interfacing with an external device.

18. The method according to claim 16, wherein generating the tinnitus signal occurs independent of data received by the stimulator module from a signal processing module, the signal processing module processing an audio signal representative of environmental sound to form a processed audio signal for providing to the stimulator module; the stimulator module including at least one mode where the stimulation signal is developed based, at least in part, on the processed audio signal.

19. The method according to claim 18, wherein the signal processing module is external to the user.

20. The method according to claim 18, wherein the signal processing module is implanted into the user.

21. The method according to claim 16, wherein the tinnitus suppression signal is unrelated to acoustic signals of the nearby environment.

22. The method according to claim 16, wherein the tinnitus suppression signal includes biphasic pulses for sequentially stimulating at least one electrode, the biphasic pulses are non-overlapping in time.

23. The method according to claim 16, wherein the tinnitus suppression signal includes pulses for simultaneously stimulating two or more electrodes.

24. The method according to claim 16, wherein the tinnitus suppression signal includes stimulation pulses having a time varying envelope based on a noise modulation function.

25. The method according to claim 16, wherein the tinnitus suppression signal when applied to the audio sensing tissue of the user is not significantly perceptible.

26. The method according to claim 16, wherein the tinnitus suppression signal when applied to the audio sensing tissue of the user is not significantly perceptible to the user.

27. The method according to claim 16, wherein the tinnitus suppression signal is masked by the natural signal processing of the user.

28. The method according to claim 16, where the prothesis is a brainstem implant.

29. The method according to claim 16 wherein the prosthesis is a middle ear implant.

30. The method according to claim 16, wherein the prosthesis is a bone conducting implant.

31. A computer program product in a computer readable storage medium, the product including program code for producing a data signal for an implanted audio prosthesis, the product comprising:
program code for generating, in a stimulator module of an implanted prosthesis, a stimulation signal for application to audio sensing tissue of a user, the stimulator module including at least a tinnitus suppression mode in which the stimulation signal includes a tinnitus suppression signal, the tinnitus suppression signal generated as a function of at least one parameter stored in stimulator memory, independent of any data received substantially simultaneously by the stimulator module, if any.

32. The computer program product according to claim 31, wherein the program code for generating the stimulation signal generates the tinnitus suppression signal without contemporaneously interfacing with an external device.

33. The computer program product according to claim 31, wherein the program code for generating the stimulation signal generates the tinnitus signal independent of data received by the stimulator module from a signal processing module, the signal processing module processing an audio signal representative of environmental sound to form a processed audio signal for providing to the stimulator module; the stimulator module including at least one mode where the stimulation signal is developed based, at least in part, on the processed audio signal.
